(19) United States
(12) Patent Application Publication
(10) Pub. No.: US 2026/0099972 A1
(43) Pub. Date: Apr. 9, 2026

(54) THREE-DIMENSIONAL IMAGE PROCESSING DEVICE, THREE-DIMENSIONAL IMAGE PROCESSING METHOD, AND PROGRAM

(71) Applicant: TOHOKU UNIVERSITY, Sendai-shi, Miyagi (JP)

(72) Inventors: Satoshi YAMAGUCHI, Sendai-shi, Miyagi (JP); Megumi NAKAMURA, Sendai-shi, Miyagi (JP)

(21) Appl. No.: 19/114,013

(22) PCT Filed: Sep. 27, 2022
(86) PCT No.: PCT/JP2022/035892

§ 371 Date: Mar. 21, 2025

(51) Int. Cl. G06T 12/10

(52) U.S. Cl. CPC G06T 12/10

(57) ABSTRACT

A three-dimensional image processing device for generating image data of a three-dimensional image used for an analysis of a diagnosis target, the three-dimensional image processing device including: an image processing unit configured to execute alignment between a three-dimensional image showing an analysis target captured at a first timing and a three-dimensional image showing the analysis target captured at a second timing different from the first timing, in which each of the three-dimensional images includes an image of an object in a preset space including the analysis target, the diagnosis target is present in the space, the analysis target is a support unit, and the alignment includes: first registration processing of performing a rigid transformation on one three-dimensional image of the two three-dimensional images to reduce a difference between the one three-dimensional image and the other three-dimensional image of the two three-dimensional images; and second registration processing of performing the rigid transformation on the one three-dimensional image to reduce a difference between an image of the analysis target shown in the other three-dimensional image and the image of the analysis target shown in the one three-dimensional image after execution of the first registration processing.

### TECHNICAL FIELD

The present invention relates to a three-dimensional image processing device, a three-dimensional image processing method, and a program.

### BACKGROUND ART

In an alveolar bone supporting a tooth, bone resorption (defect) may be caused due to periodontal disease, and there is a risk that the tooth will be lost at the end as the bone resorption progresses. Not only in the case of natural teeth, but also in a case of dental implants or orthopedic implants, it is said that resorption (defect) of a bone around a support unit is likely to occur.

In the related art, in the case of the alveolar bone, a qualitative method for evaluating bone resorption includes measuring a depth of a periodontal pocket (a groove between a tooth root and a gum) by a periodontal probe, examining whether bleeding occurs due to a stimulus of the measurement, and diagnosing stability of teeth using a tweezer.

However, it is difficult to accurately know a state of the alveolar bone lost by periodontal disease from results of such examinations. In addition, these examination results do not allow a patient himself or herself to easily understand the state of his or her periodontal disease. Therefore, X-ray photographs of teeth and surroundings thereof are used to capture a morphology of the alveolar bone visually.

Further, in recent years, with the spread of a dental cone beam CT, a state of a tooth or an alveolar bone around the tooth can be captured as a three-dimensional image. Such a method has an effect of reducing a burden on the body, and also has an effect of allowing a patient to easily understand an examination result because the result is obtained in a form of an image.

### CITATION LIST

### Patent Literature

PTL 1: JP2015-116303A.

### Non Patent Literature

NPL 1: V Chappuis, O Engel, M Reyes, K Shahim, L-P Nolte, D Buser, “Ridge alterations post-extraction in the esthetic zone: a 3D analysis with CBCT”, J. Dent. Res., Dec; 92(12 Suppl): 195S-201S. doi: 10.1177/0022034513506713. Epub 2013 Oct. 24. PMID: 24158340; PMCID: PMC3860068.

### SUMMARY OF INVENTION

### Technical Problem

In such an examination using a three-dimensional image for medical use, for example, what kind of change occurs may be diagnosed in comparison with a past result. By performing such a comparison, it is possible to more diversely and more accurately evaluate a bone resorption state of a region of interest, for example, progression of periodontal disease, as compared with a case where an evaluation is simply performed based on a result of single capturing.

However, the comparison is generally visual and qualitative, and a precise and quantitative comparison in units of pixels which can be performed by matching positions of images is not performed. As a result, information necessary for diagnosis is not sufficiently obtained, and an accuracy of the diagnosis may be low. In fact, there are reasons for this, and one of the reasons is that a morphological change over time in a tooth and periodontal tissues is always larger than that of hard tissues of other portions of the human body, and it is difficult to perform precise alignment necessary for comparing three-dimensional images captured at different times.

Although there are such circumstances, in order to reduce time, money, and a load on the body, it is desirable to obtain more quantitative information and further improve the accuracy of diagnosis. Note that such circumstances are not limited to people, and the same applies to animals. In addition, such circumstances are the same for dental implants or orthopedic implants other than a natural tooth.

In view of the above circumstances, an object of the present invention is to provide a technique of improving an accuracy of diagnosis using a three-dimensional image.

### Solution to Problem

An aspect of the present invention relates to a three-dimensional image processing device for generating image data of a three-dimensional image used for an analysis of a diagnosis target, the three-dimensional image processing device including: an image processing unit configured to execute alignment between a three-dimensional image showing an analysis target captured at a first timing and a three-dimensional image showing the analysis target captured at a second timing different from the first timing, in which each of the three-dimensional images includes an image of an object in a preset space including the analysis target, the diagnosis target is present in the space, the analysis target is a support unit, and the alignment includes: first registration processing of performing a rigid transformation on one three-dimensional image of the two three-dimensional images to reduce a difference between the one three-dimensional image and the other three-dimensional image of the two three-dimensional images; and second registration processing of performing the rigid transformation on the one three-dimensional image to reduce a difference between an image of the analysis target shown in the other three-dimensional image and the image of the analysis target shown in the one three-dimensional image after execution of the first registration processing.

An aspect of the present invention relates to a three-dimensional image processing method for generating image data of a three-dimensional image used for an analysis of a diagnosis target, the three-dimensional image processing method including: an image processing step of executing alignment between a three-dimensional image showing an analysis target captured at a first timing and a three-dimensional image showing the analysis target captured at a second timing different from the first timing, in which each of the three-dimensional images includes an image of an object in a preset space including the analysis target, the diagnosis target is present in the space, the analysis target is a support unit, and the alignment includes: first registration processing of performing a rigid transformation on one three-dimensional image of the two three-dimensional images to reduce a difference between the one three-dimensional image and the other three-dimensional image of the two three-dimensional images; and second registration processing of performing the rigid transformation on the one three-dimensional image to reduce a difference between an image of the analysis target shown in the other three-dimensional image and the image of the analysis target shown in the one three-dimensional image after execution of the first registration processing.

An aspect of the invention is a program for causing a computer to function as the above three-dimensional image processing device.

### Advantageous Effects of Invention

According to the present invention, it is possible to improve an accuracy of diagnosis using a three-dimensional image.

### BRIEF DESCRIPTION OF DRAWINGS

FIG. 1 is an illustration diagram illustrating an overview of a three-dimensional image processing device according to an embodiment.

FIG. 2 is a diagram illustrating an example of mask data in the embodiment.

FIG. 3 is a flowchart illustrating an example of second registration processing using the mask data in the embodiment.

FIG. 4 is an example of an experimental result for evaluating three-dimensional alignment processing in the embodiment.

FIG. 5 is a diagram illustrating an example of a hardware configuration of the three-dimensional image processing device according to the embodiment.

FIG. 6 is a diagram illustrating an example of a configuration of a control unit included in the three-dimensional image processing device according to the embodiment.

FIG. 7 is a flowchart illustrating an example of a flow of processing executed by the three-dimensional image processing device according to the embodiment.

FIG. 8 is a diagram illustrating an example of an experimental result in the embodiment.

### DESCRIPTION OF EMBODIMENTS

### Embodiment

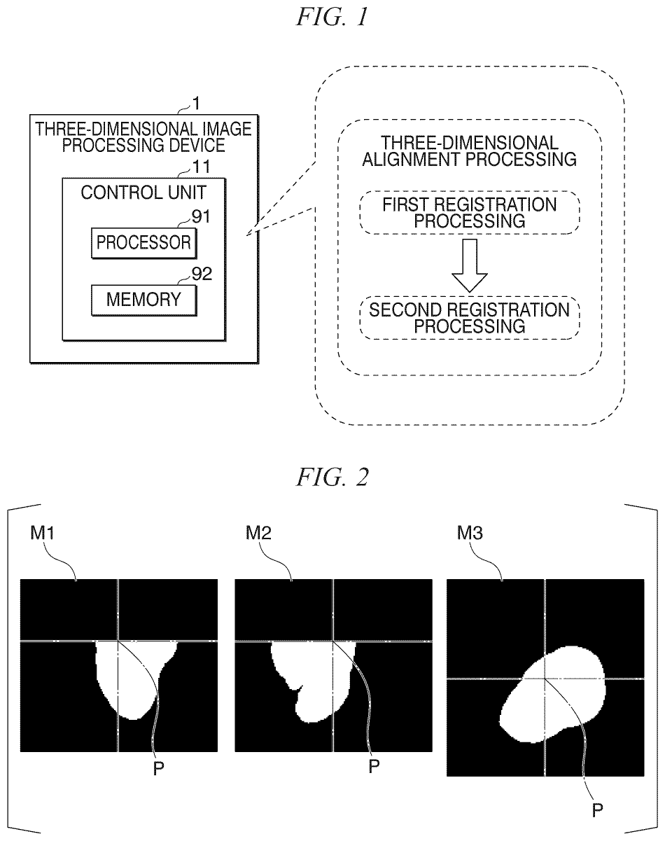

FIG. 1 is an illustration diagram illustrating an overview of a three-dimensional image processing device 1 according to an embodiment. The three-dimensional image processing device 1 generates image data of a three-dimensional image used for an analysis of a diagnosis target. The diagnosis target is, for example, a tissue around a tooth root of a natural tooth. The diagnosis target is, for example, a tissue around a fixture and an abutment of a dental implant. The diagnosis target is, for example, a tissue around a holding unit of an orthopedic implant.

Hereinafter, the tooth root of the natural tooth, the fixture and abutment of the dental implant, and the holding unit of the orthopedic implant are collectively referred to as a support unit. If a description is performed with an expression of the support unit, the diagnosis target is, for example, a tissue around the support unit. The tissue around the support unit which is the diagnosis target is, for example, a tissue that contributes to support of the support unit to a predetermined degree or more. Therefore, the tissue around the support unit is, for example, a periodontal tissue. The tissue around the support unit may be, for example, an alveolar bone or a femur.

Note that the natural tooth includes a tooth crown and the tooth root. The dental implant includes an upper structure corresponding to the tooth crown of the natural tooth, the fixture supporting the upper structure, and the abutment connecting the fixture and the upper structure. The orthopedic implant includes a head or a plate that functions as a joint, and a holding unit including a stem or a screw that supports the head or the plate.

Hereinafter, the tooth crown of the natural tooth, the upper structure of the dental implant, and the head and the plate of the orthopedic implant are collectively referred to as a functional unit.

The natural tooth may be a human natural tooth or an animal natural tooth. The dental implant may be a human dental implant or an animal dental implant. The orthopedic implant may be a human orthopedic implant or an animal orthopedic implant.

The three-dimensional image processing device 1 includes a control unit 11. The control unit 11, which will be described in detail later, includes a processor 91 such as a central processing unit (CPU) and a memory 92, and executes various types of processing by operations of the processor 91 and the memory 92.

The control unit 11 executes three-dimensional alignment processing on an analysis target image and a comparison target image. The three-dimensional alignment processing is processing of performing alignment between two three-dimensional images as execution targets. The execution targets are the analysis target image and the comparison target image.

The analysis target image is a three-dimensional image showing an image of an analysis target at a first timing. The comparison target image is a three-dimensional image showing an image of the analysis target at a second timing which is a timing different from the first timing. More specifically, the analysis target image is a three-dimensional image showing the analysis target captured at the first timing, and the comparison target image is a three-dimensional image showing the analysis target captured at the second timing different from the first timing.

More specifically, a three-dimensional image showing a result of capturing, at the second timing, the analysis target captured at the first timing is the comparison target image, and a three-dimensional image showing the analysis target captured at the first timing is the analysis target image. Conversely, a three-dimensional image showing a result of capturing, at the first timing, the analysis target captured at the second timing is the analysis target image, and the three-dimensional image showing the analysis target captured at the second timing is the comparison target image.

Note that the second timing is, for example, a timing earlier than the first timing. The second timing may be a timing later than the first timing. Hereinafter, for simplicity of description, a case where the first timing is later than the second timing will be described as an example.

Although it has been described that the analysis target image and the comparison target image are three-dimensional images each showing the image of the analysis target, more specifically, each of the analysis target image and the comparison target image shows an image of an object in a preset space (hereinafter, referred to as “image captured space”) including target. The diagnosis target is present in the image captured space. Therefore, each of the analysis target image and the comparison target image shows the image of the diagnosis target. More specifically, the analysis target is the support unit. Each of the analysis target image and the comparison target image may further include an image of the functional unit. That is, the functional unit may be present in the image captured space. The image captured space is a so-called region of interest.

The image captured space may be a range defined according to a predetermined rule, or may be a range defined by a user, for example. The predetermined rule may be any rule as long as the analysis target and the diagnosis target are included in the image captured space. The predetermined rule may be, for example, a rule that a spherical space having a predetermined radius centered on the analysis target and including the diagnosis target is defined as the image captured space.

An example of the predetermined rule in a case where there is a known deviation in morphology or a positional relationship between the analysis target and the diagnosis target will be described. The predetermined rule in such a case may be, for example, a rule that the image captured space is defined by a cuboid in which a square formed by sides having a predetermined length centered on a point a specified distance above an upper end of the analysis target is set as an upper surface, the diagnosis target is included, and a specified height is set.

As described above, the three-dimensional image showing a result of capturing, at the second timing, the analysis target captured at the first timing is the comparison target image, and the three-dimensional image showing the analysis target captured at the first timing is the analysis target image. Therefore, a person or an animal having the analysis target in the analysis target image is the same as the person or the animal having the analysis target in the comparison target image.

Hereinafter, for simplicity of description, the three-dimensional image processing device 1 will be described using an example in which the analysis target is a tooth root.

Specifically, the three-dimensional alignment processing is the processing of performing the rigid transformation on the comparison target image of the two images as the execution targets to reduce a difference between a first tooth root image and a second tooth root image. The first tooth root image is an image of the analysis target shown in the analysis target image. The second tooth root image is an image of the analysis target shown in the comparison target image.

The processing of performing the rigid transformation on the comparison target image to reduce the difference between the first tooth root image and the second tooth root image is, for example, a transformation of increasing a sum of mutual information amount between pixel values located at the same coordinates on the two images as the execution targets.

As described above, the analysis target image and the comparison target image as the execution targets of the three-dimensional alignment processing are images each showing an image of a tooth of the same person or animal, but capturing timings are different. One three-dimensional image of such two three-dimensional images (that is, the analysis target image and the comparison target image) is, for example, an image showing images of the analysis target and a tooth or an alveolar bone around the analysis target. In such a case, the other three-dimensional image is, for example, an image showing images of the analysis target of the same person or animal and a tooth or an alveolar bone around the analysis target, and is an image in which a part of the tooth around the analysis target is lost due to tooth extraction. The other three-dimensional image may be, for example, an image showing images of the analysis target of the same person or animal or a tooth around the analysis target and an image of an alveolar bone partially absorbed and becoming defective.

Incidentally, the analysis target image and the comparison target image are, for example, images showing a part of images obtained as a result of capturing by an image capturing device such as an X-ray device, and images showing in the region of interest designated by a user. Hereinafter, processing of generating, based on the image obtained as a result of capturing by the image capturing device such as an X-ray device, the image which is a part of the image obtained as a result of capturing by the image capturing device and shown in the region of interest is referred to as pre-image forming processing.

The pre-image forming processing may be performed by the user using another computer before image data of the analysis target image and image data of the comparison target image are input to the three-dimensional image processing device 1, for example. The designation of the analysis target may be executed by the control unit 11 in accordance with an instruction of the user via an input unit 12 to be described later after the image data of the analysis target image and the image data of the comparison target image are input to the three-dimensional image processing device 1.

In the designation of the analysis target, for example, processing according to information input by the user is executed. Specifically, the information input by the user and used to designate the analysis target is information indicating one point in the image and between the tooth crown and the tooth root as the analysis targets.

Hereinafter, information that is used to designate the analysis target and indicates the one point in the image and between the tooth root, which is the analysis target, and the tooth crown of the tooth having the analysis target is referred to as first designation information. Note that the one point between the tooth root, which is the analysis target, and the tooth crown of the tooth having the analysis target means one point that is a location where the tooth root, which is the analysis target, and the tooth crown of the tooth having the analysis target can be distinguished from each other. An object is to input information to an analysis device that a tooth root position is in an upward direction from the designated point when the upper jaw is designated, and the tooth root position is in a downward direction from the designated point when the lower jaw is designated.

In the designation of the analysis target, a region having a preset predetermined shape and magnitude determined with reference to the one point indicated by such first designation information is set as the region of interest. Therefore, in the designation of the analysis target, for example, a region having a preset predetermined shape and magnitude centered on the one point indicated by the first designation information is set as the region of interest.

The image data of the two images, that is, the analysis target image and the comparison target image as the execution targets of the three-dimensional alignment processing is, for example, image data of images obtained by such pre-image shaping processing. Note that the pre-image shaping processing does not necessarily have to be executed, and the image obtained as a result of capturing by the image capturing device may be used as it is as the execution target of the three-dimensional alignment processing.

Hereinafter, a set of the two three-dimensional images as the execution targets of the three-dimensional alignment processing is referred to as a target image set. That is, the target image set is a set of the analysis target image and the comparison target image.

Hereinafter, for simplicity of description, the three-dimensional image processing device 1 will be described using an example in which the analysis target image and the comparison target image included in the target image set are images of the same person captured at different timings, and the comparison target image is an image before tooth extraction and the analysis target image is an image after tooth extraction.

Note that, between the two three-dimensional images of the target image set, there is not necessarily a drastic change such as a defect of a surrounding tooth due to tooth extraction. Regardless of the presence or absence of tooth extraction, diagnosis using the three-dimensional image obtained by the three-dimensional image processing device 1 is possible if the analysis target is included in both images of the target image set. The diagnosis is, for example, an analysis of morphological changes of the alveolar bone or the like around the analysis target.

Note that the image data indicating the analysis target image and the image data indicating the comparison target image satisfy a same size condition until the processing of generating the three-dimensional emphasized display image is performed. The same size condition is a condition that magnitude of sizes in respective dimensions of the analysis target image is the same as magnitude of sizes in respective corresponding dimensions of the comparison target image.

That is, the same size condition is a condition that the sizes (x, y, z) of the analysis target image and the sizes (x′, y′, z′) of the comparison target image have a relationship of x=x′, y=y′, and z=z′. As described above, the same size condition is a condition that each of the three-dimensional dimensions is the same as the magnitude of the corresponding dimension of the three-dimensional image of the comparison target.

Note that x represents magnitude of a first dimension of the analysis target image, which is a three-dimensional image. y represents magnitude of a second dimension of the analysis target image which is a three-dimensional image. z represents magnitude of a third dimension of the analysis target image which: a three-dimensional image. x represents magnitude of a first dimension of the comparison target image which is a three-dimensional image. y′ represents magnitude of a second dimension of the comparison target image which is a three-dimensional image. Z represents magnitude of a third dimension of the comparison target image which is a three-dimensional image.

When the above-described pre-image forming processing is executed, the analysis target image and the comparison target image as the execution targets of the three dimensional alignment processing satisfy the same size condition. This is because the shape and magnitude of the region of interest are preset as described above, and the region of interest having the same shape and magnitude is set in the pre-image forming processing regardless of the image.

Regarding the magnitude of the entire image in XYZ directions, when the magnitude of the analysis target image is adjusted to fit the region of interest, the comparison target image is slightly larger than the analysis target image, and then cropped to fit the analysis target image after a first registration, missing parts in the comparison target image after the first registration can be further prevented from occurring. This is because an angle transformation causes a part that is originally outside the image to be included in the transformed image when the magnitude is the same.

The three-dimensional alignment processing includes first registration processing and second registration processing. The first registration processing is processing of performing a rigid transformation on the comparison target image to reduce a difference between entire images included in the target image set. That is, the first registration processing is the processing of performing the rigid transformation on one three-dimensional image of the two three-dimensional images, that is, the analysis target image and the comparison target image to reduce a difference between the one three-dimensional image and the other three-dimensional image. Hereinafter, the transformed comparison target image transformed by the first registration processing is referred to as a first transformed image.

In the first registration processing, one point on the comparison target image and one point on the analysis target image may be designated. In such a case, the rigid transformation is performed on the comparison target image to reduce the difference between the comparison target image and the analysis target image from a state in which the designated one points of the images are matched.

Note that in the first registration, when the magnitude of the images is different, processing of matching points designated by the user between the tooth crown and the tooth root may be executed.

Hereinafter, information that is used for the first registration processing and designates one point on the comparison target image and one point on the analysis target image is referred to as second designation information. The one point indicated by the second designation information is, for example, one point between the tooth root, which is the analysis target, and the tooth crown of the tooth having the analysis target.

The second designation information is input to the three-dimensional image processing device 1 by the user via the input unit 12 to be described later, for example. The control unit 11 acquires the second designation information input by the user, and performs the rigid transformation to reduce the difference between the comparison target image and the analysis target image in a state in which the points indicated by the acquired second designation information are matched.

Note that the points indicated by the second designation information may be the same as the point indicated by the first designation information. When the control unit 11 executes the pre-image shaping processing, input of the first designation information to the three-dimensional image processing device 1 is completed before the execution of the first registration processing. Therefore, in such a case, the first designation information may be used as the second designation information.

The second registration processing is processing of performing the rigid transformation on the first transformed image to reduce a difference between an image of the analysis target shown in the first transformed image and an image of the analysis target shown in the analysis target image.

The second registration processing is, for example, processing of performing a transformation on the first transformed image to reduce, based on the information indicating the image of the analysis target shown in the images, a difference in morphology of the image of the analysis target indicated by the information. The second registration processing may be, for example, processing of performing the rigid transformation obtained in accordance with the predetermined rule on the first transformed image. An example of processing in accordance with such a predetermined rule is processing using the mask data to be described later, for example. An example of the second registration processing using the mask data will be described later.

### Significance of Reducing Difference in Morphology of Tooth Root Shown in Image

Morphological changes over time in teeth and periodontal tissues are more rapid than those of other parts of the human body. However, among the teeth or periodontal tissues, the tooth root is a tissue whose morphological change over time is relatively small. Also in a dental implant, a functional unit may be replaced, a morphological change of a surrounding bone is large, and a morphological change of a support unit is small. In an orthopedic implant, a morphology of the support unit is less likely to change compared to a morphology of the surrounding bone.

Therefore, if the change of the teeth or the periodontal tissues is estimated with reference to a position of the tooth root, it is possible to estimate the change of the teeth or the periodontal tissues with higher accuracy as compared with a case of estimating the change with reference to other portions. Therefore, the control unit 11 can estimate the change of the teeth or periodontal tissues with higher accuracy by executing the processing of reducing the difference in the morphology between the image of the tooth root shown in the analysis target image and the image of the tooth root shown in the comparison target image. Also in the dental implant or the orthopedic implant, it is possible to estimate the change of the surrounding tissue with high accuracy by estimating the change in the surrounding tissue with reference to the position of the support unit.

### Significance of Executing First Registration Processing

Execution of the first registration processing before the execution of the second registration processing prevents occurrence of a situation such as overlearning in machine learning. Specifically, the execution of the first registration processing before the execution of the second registration processing prevents occurrence of a situation where a degree of matching between the two images is high only in the periphery of the image of the tooth root indicated by tooth root designation information and is low in the entire image.

### Details of Second Registration Processing Using Mask Data

In the second registration processing, the mask data may be used. The mask data is data indicating a pixel of an unchanged tooth root inclusion region (hereinafter, referred to as “tooth root inclusion pixel”) among the pixels of the analysis target image. The unchanged tooth root inclusion region is a region on the analysis target image and is a region including the image of the analysis target.

The mask data is, for example, image data of a binary image (hereinafter, referred to as a “mask image”) satisfying a mask image condition. The mask image condition is a condition that a pixel value of the unchanged tooth root inclusion region is one pixel value of two preset pixel values and a pixel value of a region other than the unchanged tooth root inclusion region is the other pixel value of the two preset pixel values.

The preset two pixel values are, for example, 0 and 1. A size of the mask image is the same as those of the analysis target image and the first transformed image. Therefore, the mask image is a three-dimensional image. The mask data may be, for example, information indicating a boundary of the unchanged tooth root inclusion region.

Note that the mask data is, for example, image data of a binary image in which an image of an object in a predetermined space including the analysis target and other images have different pixel values. The predetermined space including the analysis target is, for example, a space obtained by enlarging a space matching the image of the analysis target by a predetermined width in all directions.

FIG. 2 is a diagram illustrating an example of the mask data in the embodiment. More specifically, FIG. 2 is a diagram illustrating an example of the mask image in a case where the mask data in the embodiment is the image data of the mask image. FIG. 2 shows an image M1, an image M2, and an image M3.

The image M1 is a diagram of the mask image viewed from a direction of one of three axes orthogonal to each other (hereinafter, referred to as a “first axial direction”). The image M2 is a diagram of the mask image viewed from a direction of another one of the three axes orthogonal to each other (hereinafter, referred to as a “second axial direction”). The image M3 is a diagram of the mask image viewed from a direction of one of the three axes orthogonal to each other, that is, a direction of a vector perpendicular to a vector parallel to the first axial direction and perpendicular to a vector parallel to the second axial direction (hereinafter, referred to as a “third axial direction”).

A point P in FIG. 2 is an example of the one point in the three-dimensional image indicated by the second designation information.

Accordingly, the mask data is information that indicates for each pixel whether to be an unchanged tooth root inclusion region. Therefore, for example, when a value of a tooth root inclusion pixel of the mask data is 1 and values of the other pixels are 0, an image including the image of the analysis target is obtained by multiplying each pixel of the analysis target image by the value of each pixel indicated by the mask data. Further, by multiplying each pixel of the first transformed image by the value of each pixel indicated by the same mask data, an image that is highly likely to include the image of the analysis target, that is, an image that has a high degree of matching with the image obtained from the analysis target image is obtained.

However, since the mask data is obtained based on the analysis target image, such an image is not necessarily obtained for the first transformed image. Therefore, if an appropriate rigid transformation is executed on the first transformed image, the first transformed image includes the image of the analysis target, and thus, an image having a high degree of matching with the image obtained from the analysis target image is obtained.

In this Way, the processing of performing the transformation on the first transformed image to reduce the difference in the morphology of the image of the analysis target is the second registration processing using the mask data. The second registration processing using the mask data will be further described.

In the second registration processing using the mask data, the rigid transformation is executed on the first transformed image to increase a degree of matching between a partial analysis target image and a partial comparison target image. The partial analysis target image is an image of the unchanged tooth root inclusion region in the analysis target image. More specifically, the partial analysis target image is an image of a part of the analysis target image acquired based on the image data of the analysis target image and the mask data, and is an image of the unchanged tooth root inclusion region.

The partial comparison target image is an image acquired based on the image data of the first transformed image and the mask data, is a region on the image of the first transformed image, and is an image of an image shown in candidate image extraction. A candidate image extraction region is a region that satisfies a condition that if an image where the region is present is not the first transformed image but the analysis target image, the region is a tooth root inclusion region.

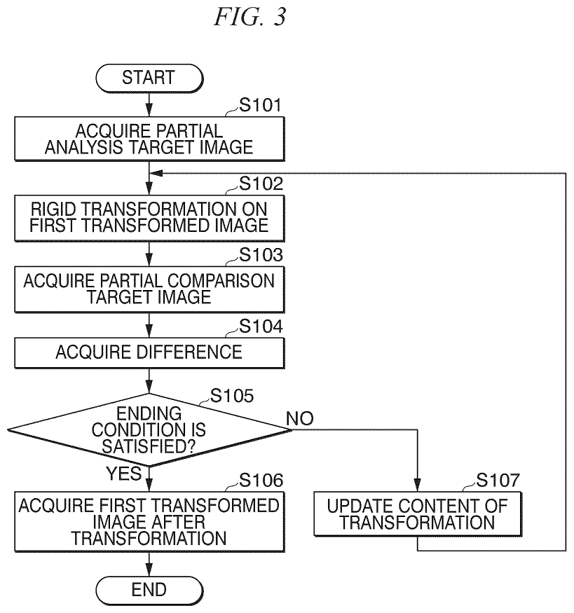

FIG. 3 is a flowchart illustrating an example of the second registration processing using the mask data in the embodiment. Note that the control unit 11 executes the processing described in FIG. 3.

In the second registration processing using the mask data, first, only a mask region is extracted from the analysis target image to acquire the partial analysis target image (step S101). In the second registration processing using the mask data, next, the rigid transformation is executed on the first transformed image (step S102).

In the second registration processing using the mask data, next, the partial comparison target image is acquired from the first transformed image after the execution of the rigid transformation (step S103). In the second registration processing using the mask data, next, a difference between the obtained partial analysis target image and the obtained partial comparison target image is acquired (step S104).

In the second registration processing using the mask data, next, it is determined whether a predetermined ending condition related to smallness of the difference obtained in step S104 is satisfied (step S105). The predetermined ending condition may be, for example, a condition that the difference is smaller than a predetermined difference. The predetermined ending condition may be, for example, a condition that the difference converges to be smaller than the predetermined difference.

The predetermined ending condition may be, for example, a condition that mutual information amount between the partial analysis target image and the partial comparison target image converges. Since the mutual information amount between the partial analysis target image and the partial comparison target image is an amount indicating the degree of matching between the partial analysis target image and the partial comparison target image, convergence of the mutual information amount between the partial analysis target image and the partial comparison target image means convergence of the difference.

If the ending condition is not satisfied (step S105: NO), a content of the rigid transformation is updated in accordance with the predetermined rule to reduce the difference between the partial analysis target image and the partial comparison target image (step S107).

Specifically, a value of a parameter that determines the content of the rigid transformation is updated in accordance with the predetermined rule to reduce the difference between the partial analysis target image and the partial comparison target image. After step S107, the processing returns to step S102.

On the other hand, if the ending condition is satisfied (step S105: YES), the first transformed image transformed by the processing of the immediately preceding step S102 is obtained as a result of the second registration processing (step S106). After the execution of step S106, the processing ends.

In this way, in the second registration processing, the first transformed image satisfying the condition that the difference between the image of the analysis target shown in the second transformed image and the image of the analysis target shown in the analysis target image is smaller than that before the execution of the second registration processing is obtained. More specifically, in the second registration processing, the first transformed image in which the difference between a position or an inclination of the image of the analysis target shown in the first changed image and a position or an inclination of the image of the analysis target shown in the analysis target image is smaller than that before the execution of the second registration processing is obtained.

Hereinafter, the first changed image transformed by executing the second registration processing is referred to as the second transformed image. Therefore, an image obtained as a result of the second registration processing is the second transformed image.

The difference between the morphology of the image of the analysis target shown in the analysis target image and the morphology of the image of the analysis target shown in the second transformed image is smaller than the difference between the morphology of the image of the analysis target shown in the analysis target image and the morphology of the image of the analysis target shown in the first transformed image. Since the morphological change over time of the support unit such as the tooth root is smaller than that of other periodontal tissues, a state change over time of the tooth or the periodontal tissue generated between the analysis target image and the second transformed image can be estimated with higher accuracy if the analysis target image and the second transformed image are used.

### Experimental Result

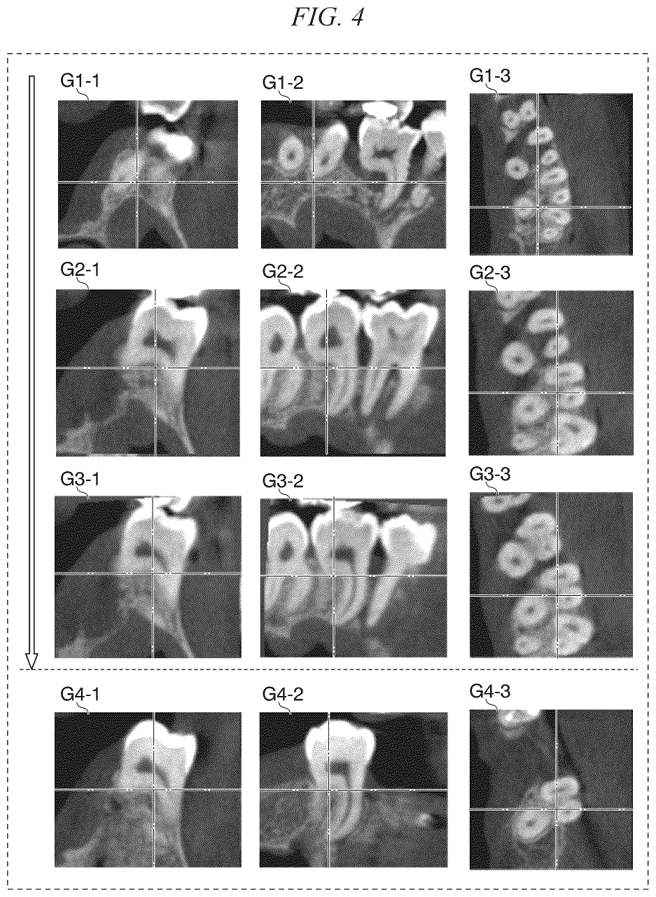

FIG. 4 is an example of an experimental result for evaluating the three-dimensional alignment processing in the embodiment. FIG. 4 illustrates an image G1-1, an image G1-2, an image G1-3, an image G2-1, an image G2-2, an image G2-3, an image G3-1, an image G3-2, an image G3-3, an image G4-1, an image G4-2, and an image G4-3.

The image G1-1 is a diagram of a before-transformation target image viewed from the first axial direction. The before-transformation target image is a comparison target image before the execution of the three-dimensional alignment processing. The image G-2 is a diagram of the before-transformation target image viewed from the second axial direction. The image G1-3 is a diagram of the before-transformation target image viewed from the third axial direction.

The image G2-1 is a diagram of the first transformed image viewed from the first axial direction. The image G2-2 is a diagram of the first transformed image viewed from the second axial direction. The image G2-3 is a diagram of the first transformed image viewed from the third axial direction. The image G3-1 is a diagram of the second transformed image viewed from the first axial direction. The image G3-2 is a diagram of the second transformed image viewed from the second axial direction. The image G3-3 is a diagram of the second transformed image viewed from the third axial direction.

The image G4-1 is a diagram of the analysis target image viewed from the first axial direction. The image G4-2 is a diagram of the analysis target image viewed from the second axial direction. The image G4-3 is a diagram of the analysis target image viewed from the third axial direction.

FIG. 4 illustrates that a first transformed target image has a smaller difference from the analysis target image in the position or the inclination of the analysis target than the before-transformation target image, and the second transformed image has a smaller difference from the analysis target image in the position or the inclination of the analysis target than the first transformed image. Accordingly, FIG. 4 illustrates that a comparison target image having a small difference in the position or the inclination of the analysis target from the analysis target image is obtained by the three-dimensional alignment processing.

Accordingly, the three-dimensional alignment processing is alignment processing for two three-dimensional images.

Both the analysis target image and the second transformed image are three-dimensional images. Therefore, it is possible to generate a three-dimensional image (hereinafter, referred to as a “three-dimensional emphasized display image”) in which the difference between the analysis target image and the second transformed image is indicated by emphasis display such as coloring. Such processing of generating the three-dimensional emphasized display image (hereinafter, referred to as “three-dimensional emphasized display image generation processing”) is executed by, for example, the control unit 11.

If the three-dimensional emphasized display image is generated, the user can visually know a difference between a state of the tooth or periodontal tissue shown in the analysis target image and a state of the tooth or periodontal tissue shown in the comparison target image.

Generation of the three-dimensional emphasized display image is useful not only for medical specialists but also for patients who are laypeople in the medical field. This is because knowledge about a content of each numerical value is required because a chart of a periodontal tissue examination is a list of numerical values, but in the case of the three-dimensional image, such knowledge becomes less necessary, and thus it is easy to know the examination result.

Note that the three-dimensional emphasized display image may be, for example, a three-dimensional image in which a difference between a positive difference and a negative difference is indicated by different colors.

Note that the three-dimensional emphasized display image may be, for example, an image that indicates that the tooth root belongs to a first tooth root portion, a second tooth root portion, or a third tooth root portion. The first tooth root portion is a part of the tooth root and is not covered by a bone at a first time point. The second tooth root portion is a part of the tooth root, and is covered by the bone at the first time point but is not covered by the bone at a second time point that is later than the first time point. The third tooth root portion is a part of the tooth root and is covered by the bone at both the first time point and the second time point.

In such a three-dimensional emphasized display image, the first tooth root portion is shown in white, for example, the second tooth root portion is shown in red, for example, and the third tooth root portion is shown in green, for example.

Hereinafter, the tooth or the periodontal tissue shown in the comparison target image is referred to as a first image captured tissue. Hereinafter, the tooth or the periodontal tissue shown in the analysis target image is referred to as a second image captured tissue.

Both the analysis target image and the second transformed image are three-dimensional images. Therefore, both the analysis target image and the second transformed image are a set of pixel values. Since the pixels are ordered sets, quantitative information related to the analysis target image and the second transformed image can also be acquired based on the analysis target image and the second transformed image. Hereinafter, processing of acquiring the quantitative information related to the analysis target image and the second transformed image is referred to as quantitative information acquisition processing. The quantitative information acquisition processing is performed by the control unit 11, for example.

The quantitative information related to the analysis target image and the second transformed image is, for example, information indicating the difference between the analysis target image and the second transformed image by a numerical value. The information indicating the difference between the analysis target image and the second transformed image by a numerical value is, for example, information indicating an amount of bone resorption of a second tissue with respect to a first tissue. The information indicating the difference between the analysis target image and the second transformed image by a numerical value is, for example, information indicating an amount of bone proliferation of the second tissue with respect to the first tissue.

The information indicating the difference between the analysis target image and the second transformed image by a numerical value may be, for example, information indicating a three-dimensional volume of each of the first tooth root portion, the second tooth root portion, and the third tooth root portion described above.

By using the second transformed image in this way, the quantitative information with higher can be obtained than that of using the comparison target image before the execution of the three-dimensional alignment processing. Therefore, the three-dimensional image processing device 1 that obtains the second transformed image can improve the accuracy of diagnosis of the state of the tooth or the periodontal tissue.

### Regarding Generation of Mask Data

Here, a specific example of generating the mask data will be described. Specifically, the mask data is generated by a computer. The mask data is generated by, for example, the control unit 11. Note that the generation of the mask data is not necessarily executed by the three-dimensional image processing device 1, and may be executed by another device.

In such a case, the three-dimensional image processing device 1 acquires the mask data generated by another device that generates the mask data before executing the three-dimensional alignment processing, and uses the mask data in the three-dimensional alignment processing.

Hereinafter, for simplicity of description, an example of processing of generating the mask data (hereinafter, referred to as “mask data generation processing”) will be described using, as an example, a case where the control unit 11 executes the processing.

The analysis target image is a three-dimensional image as described above. Therefore, the control unit 11 can execute the processing on the three-dimensional image as a set of two-dimensional images. In the mask data generation processing, the control unit 11 processes the analysis target image as an ordered set (hereinafter, referred to as an “analysis target ordered set”) in which analysis target slice images are elements, and a rank of the elements is ordered in a direction from the analysis target to the tooth crown of the tooth having the analysis target. Hereinafter, for simplicity of description, the direction from the analysis target toward the tooth crown of the tooth having the analysis target is referred to as a direction from the tooth root as the analysis target toward the tooth crown.

The analysis target slice images are two-dimensional images resulting from slicing the analysis target image in the direction from the tooth root as the analysis target toward the tooth crown. Therefore, the analysis target slice image is one type of a so-called slice image.

Note that the rank of the ordered set may become higher from the tooth root as the analysis target toward the tooth crown, or may become lower from the tooth crown as the analysis target toward the tooth root, and either rule is used. Note that the information on the direction from the tooth root as the analysis target to the tooth crown in the mask data processing is obtained based on, for example, point position information and jaw designation information. The point position information is information indicating one point in the image and between the tooth crown and the tooth root as the analysis targets. Both the first designation information and the second designation information described above are examples of the point position information.

The jaw designation information is information indicating whether the analysis target is an upper jaw or a lower jaw in the three-dimensional image. For example, the user inputs the jaw designation information to the three-dimensional image processing device 1 via the input unit 12 or the like. In such a case, the control unit 11 acquires the input jaw designation information. For example, the user inputs the point position information to the three-dimensional image processing device 1 via the input unit 12 or the like. In such a case, the control unit 11 acquires the input point position information.

In the mask data generation processing, the control unit 11 selects whether each analysis target slice image is a two-dimensional image for generating mask data. The two-dimensional image for generating mask data is an analysis target slice image in which a first rank difference is larger than a second rank difference, that is, an analysis target slice image in which the first rank difference is larger than an absolute value of the difference in rank between a slice image including a point indicated by the point designation information and a tooth crown boundary image.

The first rank difference is an absolute value of a difference in rank from the tooth crown boundary image. The second rank difference is an absolute value of a difference in rank from a tooth root inclusion boundary image.

The tooth crown boundary image is one analysis target slice image that satisfies a predetermined condition among analysis target slice images (hereinafter, referred to as “tooth crown images”) showing an image of the tooth crown. The tooth root inclusion boundary image is one analysis target slice image that satisfies a predetermined condition among the analysis target slice images (hereinafter, referred to as “tooth root inclusion images”) showing an image of the tooth root.

The predetermined condition satisfied by the tooth crown boundary image is, for example, a condition that a difference in rank from the tooth root inclusion boundary image is smaller than that of other tooth crown images. The predetermined condition satisfied by the tooth root inclusion boundary image is, for example, a condition that the difference in rank from the tooth crown boundary image is smaller than that of other tooth root inclusion images. Therefore, the tooth crown image may be, for example, an analysis target slice image that shows an image of the tooth crown but does not show an image of the tooth root. The tooth root inclusion image may be, for example, an analysis target slice image that shows the image of the tooth root but does not show the image of the tooth crown.

In this way, the control unit 11 obtains, as a set of two-dimensional images for generating mask data, a three-dimensional image (hereinafter, referred to as “three-dimensional image for generating mask data”) that includes the tooth root as the analysis target but does not include the tooth crown as the analysis target. Next, the control unit 11 executes binarization processing of transforming the three-dimensional image for generating mask data into a binary image in which the pixel value of the tooth image and the pixel value of other images are different.

The binarization processing includes, for example, arc-shaped connected point determination processing. The arc-shaped connected point determination processing is processing of determining that, among other ends of a curve (hereinafter, referred to as a “mask curve”) that is a curve in the three-dimensional image for generating mask data and has a point indicated by the point position information as one end, a set of the other ends of the curve satisfying an arc-shaped connection condition is an image of a tooth. That is, the arc-shaped connected point determination processing is processing of determining, among the pixels in the image as the execution target, the pixel located at the other ends of the curve (hereinafter, referred to as an “arc-shaped connected pixel”) satisfying the arc-shaped connection condition.

The arc-shaped connection condition is a condition that the difference between pixel values of all the points on the mask curve and a pixel value of the point indicated by the point position information is within a preset predetermined range.

The binarization processing including the arc-shaped connection determination processing includes setting processing. The setting processing is processing of setting one pixel value of two preset pixel values to a pixel value of an arc-shaped connected pixel and setting the other pixel value of the two preset pixel values to a pixel value of a pixel that is not the arc-shaped connected pixel. The control unit 11 can reduce a possibility that the arc-shaped connected pixel includes a region other than the tooth root, such as a bone region, by executing the arc-shaped connection determination processing to satisfy a sub-condition.

The sub-condition is a condition that the range of the arc-shaped connected pixel is smaller than an actual tooth root. After the binarization processing, the control unit 11 may execute processing of enlarging the range of the arc-shaped connected pixels by morphology transformation. The control unit 11 can generate the mask data including a safety margin around the tooth root by executing the processing of enlarging the range of the arc-shaped connected pixels by the morphology transformation after the binarization processing.

By such binarization processing, the three-dimensional image for generating mask data is transformed into the binary image in which the pixel value of the tooth image and the pixel value of the other images are different. The image data of the binarized three-dimensional image for generating mask data after the transformation is an example of the mask data.

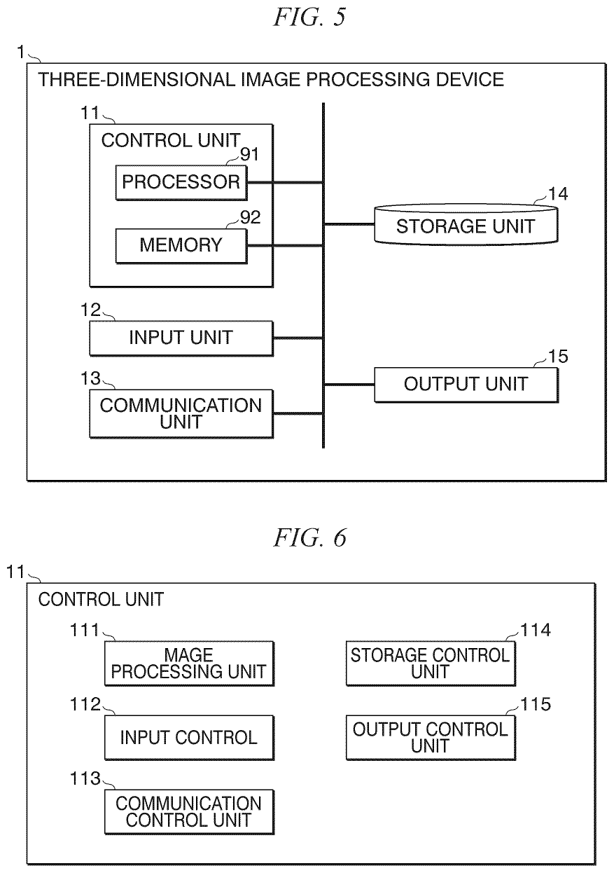

FIG. 5 is a diagram illustrating an example of a hardware configuration of the three-dimensional image processing device 1 according to the embodiment. The three-dimensional image processing device 1 includes the control unit 11 including the processor 91 such as a central processing unit (CPU) and the memory 92 connected by a bus, and executes a program. The three-dimensional image processing device 1 functions as a device including the control unit 11, the input unit 12, a communication unit 13, a storage unit 14, and an output unit 15 by executing a program.

More specifically, the processor 91 reads a program stored in the storage unit 14 and stores the read program in the memory 92. When the processor 91 executes the program stored in the memory 92, the three-dimensional image processing device 1 functions as a device including the control unit 11, the input unit 12, the communication unit 13, the storage unit 14, and the output unit 15.

The control unit 11 controls operations of various functional units included in the three-dimensional image processing device 1. The control unit 11 executes, for example, the three-dimensional alignment processing. The control unit 11 may execute, for example, the mask data generation processing. The control unit 11 may execute, for example, the three-dimensional emphasized display image generation processing. The control unit 11 may execute, for example, the quantitative information acquisition processing. The control unit 11 may execute, for example, the pre-image forming processing.

The input unit 12 includes an input device such as a mouse, a keyboard, or a touch panel. The input unit 12 may be configured as an interface that connects the input device to the three-dimensional image processing device 1. The input unit 12 receives input of various types of information to the three-dimensional image processing device 1.

For example, a user inputs information such as an instruction to the control unit 11 to the input unit 12. For example, the first designation information may be input to the input unit 12. For example, the second designation information may be input to the input unit 12. For example, the jaw designation information may be input to the input unit 12. For example, the point position information may be input to the input unit 12.

The communication unit 13 includes a communication interface for connecting the three-dimensional image processing device 1 to an external device. The communication unit 13 communicates with an external device in a wired or wireless manner. The external device is, for example, a device that is a transmission source of the analysis target image. The communication unit 13 acquires the analysis target image by communicating with the device that is the transmission source of the analysis target image. The external device is, for example, a device that is a transmission source of the comparison target image. The communication unit 13 acquires the comparison target image by communicating with the device that is the transmission source of the comparison target image.

The devices that are transmission sources of the analysis target image and the comparison target image may be the same. In such a case, the devices that are transmission sources of the analysis target image and the comparison target image may execute, for example, the pre-image forming processing. In such a case, the analysis target image and the comparison target image transmitted by the devices that are transmission sources of the analysis target image and the comparison target image are the analysis target image and the comparison target image obtained by the pre-image forming processing.

The external device may be, for example, a device that is an output destination of the image data of the second transformed image. In such a case, the communication unit 13 outputs the image data of the second transformed image to the device that is the output destination of the image data of the second transformed image by communicating with the device that is the output destination of the image data of the second transformed image.

The external device may be, for example, a device that is an output destination of the image data of the three-dimensional emphasized display image. In such a case, the communication unit 13 outputs the image data of the three-dimensional emphasized display image to the device of the output destination of the image data of the three-dimensional emphasized display image by communicating with the device of the output destination of the image data of the three-dimensional emphasized display image.

The external device may be, for example, a device that is an output destination of the quantitative information. In such a case, the communication unit 13 outputs the quantitative information to the device that is the output destination of the quantitative information by communicating with the device that is the output destination of the quantitative information.

The external device may be, for example, a device that is a generation source of the mask data. In such a case, the communication unit 13 acquires the mask data by communicating with the device that is the generation source of the mask data. The device that is the generation source of the mask data is a device that acquires the analysis target image as the execution target of the three-dimensional alignment processing, executes the mask data generation processing based on the acquired analysis target image, and generates the mask data.

The storage unit 14 is implemented by a computer-readable storage medium device such as a magnetic hard disk device or a semiconductor storage device. The storage unit 14 stores various types of information related to the three-dimensional image processing device 1. The storage unit 14 stores, for example, information input via the input unit 12 or the communication unit 13. The storage unit 14 stores, for example, the comparison target image. The comparison target image may be obtained from the external device or the like, or may be stored in advance in the storage unit 14. The storage unit 14 may store the mask data.

The output unit 15 Outputs various types of information. The output unit 15 includes a display device such as a cathode ray tube (CRT) display, a liquid crystal display, or an organic electro-luminescence (EL) display. The output unit 15 may be configured as an interface that connects these display devices to the three-dimensional image processing device 1. The output unit 15 outputs, for example, information input to the input unit 12 or the communication unit 13.

The output unit 15 may display, for example, the second transformed image. The output unit 15 may display, for example, the analysis target image. The output unit 15 may display, for example, the three-dimensional emphasized display image. The output unit 15 may display, for example, the quantitative information.

FIG. 6 is a diagram illustrating an example of a configuration of the control unit 11 included in the three-dimensional image processing device 1 according to the embodiment. The control unit 11 includes an image processing unit 111, an input control unit 112, a communication control unit 113, a storage control unit 114, and an output control unit 115.

The image processing unit 111 executes at least the three-dimensional alignment processing. The image processing unit 111 may execute, for example, the three-dimensional emphasized display image generation processing. The image processing unit 111 may execute, for example, the quantitative information acquisition processing. The image processing unit 111 may execute, for example, the pre-image forming processing. The image processing unit 111 may execute, for example, the mask data generation processing.

The image processing unit 111 may control, for example, an operation of the communication control unit 113 to cause the communication unit 13 to output the image data of the second transformed image to the device that is the output destination of the image data of the second transformed image. In such a case, the image processing unit 111 outputs the image data of the second transformed image to the communication control unit 113. The communication control unit 113 causes the communication unit 13 to output the acquired image data.

The image processing unit 111 may control, for example, an operation of the output control unit 115 to cause the output unit 15 to output the image data of the second transformed image. In such a case, the image processing unit 111 outputs the image data of the second transformed image to the output control unit 115. The output control unit 115 causes the output unit 15 to output the acquired image data.

The input control unit 112 controls an operation of the input unit 12. The communication control unit 113 controls an operation of the communication unit 13. The storage control unit 114 controls an operation of the storage unit 14.

The output control unit 115 controls an operation of the output unit 15. The output control unit 115 controls, for example, the operation of the output unit 15 to display the analysis target image on the output unit 15. The output control unit 115 controls, for example, the operation of the output unit 15 to display the second transformed image obtained by the image processing unit 111 on the output unit 15.

For example, when the image processing unit 111 executes the three-dimensional emphasized display image generation processing, the output control unit 115 may control the operation of the output unit 15 to display the three-dimensional emphasized display image obtained by the image processing unit 111 on the output unit 15. For example, when the image processing unit 111 executes the quantitative information acquisition processing, the output control unit 115 may control the operation of the unit 15 to display the quantitative information obtained by the image processing unit 111 on the output unit 15.

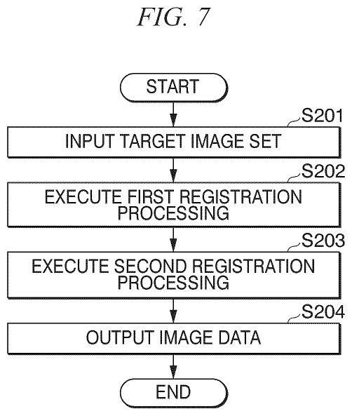

An example of a flow of processing executed by the three-dimensional image processing device 1 will be described below with reference to FIG. 7. In FIG. 7, for simplicity, an example of the processing will be described using, as an example, a case where the input of the second designation information is completed by the user in advance and the analysis target image and the comparison target image on which the execution of the pre-image forming processing is completed in advance are used.

FIG. 7 is a flowchart illustrating an example of a flow of the processing executed by the three-dimensional image processing device 1 according to the embodiment. The target image set is input to the input unit 12 or the communication unit 13 (step S201). Next, the image processing unit 111 executes the first registration processing on the images of the input target image set (step S202). Next, the image processing unit 111 executes the second registration processing (step S203).

Next, the image processing unit 111 controls the operation of the communication control unit 113 or the output control unit 115 to output the image data of the second transformed image to the output destination corresponding to each control target (step S204). Therefore, in step S204, when the image processing unit 111 controls the operation of the communication control unit 113, the image processing unit 111 controls the operation of the communication unit 13 via the control of the operation of the communication control unit 113 to output the image data of the second transformed image to the device that is the output destination. In this case, as described above, the image processing unit 111 outputs the image data of the second transformed image to the communication control unit 113.

In step S204, when the image processing unit 111 controls the operation of the output control unit 115, the image processing unit 111 controls the operation of the output control unit 115 to cause the output unit 15 to output the image data of the second transformed image. In this case, as described above, the image processing unit 111 outputs the image data of the second transformed image to the output control unit 115.

### Experimental Result

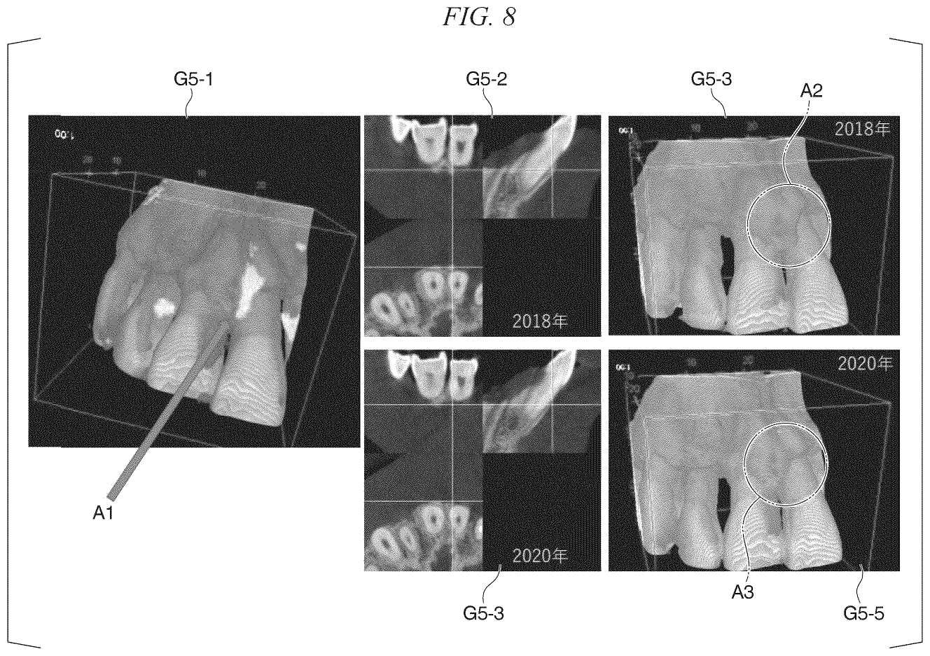

An example of a result of an experiment using the three-dimensional image processing device 1 will be described. FIG. 8 is a diagram illustrating an example of the experimental result in the embodiment. In the experiment, an image indicating a change in an alveolar bone of a patient was obtained by the three-dimensional image processing device 1. In the experiment, the image indicating the change in the alveolar bone was obtained based on an image of a tooth of a patient captured in 2018 and an image of the tooth of the patient captured in 2020. An image G5-1 shows a change in the alveolar bone indicated by emphasis display. For example, a region A1 in the image G5-1 indicates the change in the alveolar bone. An image G5-2 is an example of a cross section of a three-dimensional image in which images on the upper left, upper right, and lower left were captured in 2018.

An image G5-3 is an example of a three-dimensional image captured in 2018. An image G5-4 is an example of a cross section of a three-dimensional image in which images on the upper left, upper right, and lower left were captured in 2020. An image G5-5 is an example of a three-dimensional image captured in 2020. A difference between an image of a region A2 in the image G5-3 and an image of a region A3 in the image G5-5 is the region A1. More specifically, the region A1 indicates that resorption of the alveolar bone occurs between 2018 and 2020. According to the image G5-1, an amount of the resorption of the alveolar bone in the region A1 from 2018 to 2020 is 6.9 cubic millimeters.

In the three-dimensional image processing device 1 according to the embodiment configured in this way, the rigid transformation for reducing the difference between the analysis target images shown in the two three-dimensional images captured at different timings is performed on one three-dimensional image. As described above, the tooth root is less likely to morphologically change over time than the tooth and other periodontal tissues. Therefore, such a three-dimensional image processing device 1 can improve the accuracy of diagnosis of the state of the tooth or the periodontal tissue.

### Modification

Note that when the point position information and the jaw information are input to the three-dimensional image processing device 1, the image processing unit 111 may execute separation degree improvement image generation processing. The separation degree improvement image generation processing is processing of generating an image in which a degree of separation between the image of the tooth and the image of the alveolar bone shown in the analysis target image and the second transformed image is improved. Hereinafter, for simplicity of description, an image as an execution target of the separation degree improvement image generation processing is referred to as a separation target image. The separation target image is the analysis target image or the second transformed image.

### Regarding Separation Degree Improvement Image Generation Processing

The separation degree improvement image generation processing is an example of the mask data generation processing.

In the separation degree improvement image generation processing, first sub-separation processing is executed. The first sub-separation processing is processing of selecting a slice closer to a tooth root side than the position indicated by the point position information from slice images generated as a result of slicing the separation target image in the direction from the tooth root as the analysis target to the tooth crown based on the jaw information. Whether the slice image is the image on the tooth root side is determined based on the point position information and the jaw information as described above.

Note that the processing of selecting the slice closer to the tooth root side than the position indicated by the point position information in the resolution improvement image generation processing is, for example, the above-described processing of selecting whether each analysis target slice image is a two-dimensional image for generating mask data.

Note that when the separation target image is a CT image, the separation target image is a so-called axial image. Therefore, in such a case, a long axis of the tooth is substantially orthogonal to the slice image. The slice image in a case of analyzing a largely inclined tooth may be a slice image obtained by specifying a tooth axis or a tooth cervical line by the user and re-cutting the image into a slice orthogonal to the tooth axis.

In the separation degree improvement image generation processing, next, second sub-separation processing is executed. The second sub-separation processing is processing of binarizing the separation target image by using a threshold equal to or greater than a predetermined value. The predetermined value is a value that satisfies a condition that the region on the separation target image determined as a pixel indicating a tooth by the image processing unit 111 is small even when the threshold is less than the predetermined value. By the second sub-separation processing, for example, a binary image in which the pixel values of the tooth and the alveolar bone are 1 and other pixel values are 0 is obtained.

In the separation degree improvement image generation processing, next, third sub-separation processing is executed. The third sub-separation processing is arc-shaped connected point determination processing.

In the separation degree improvement image generation processing, next, fourth sub-separation processing is executed. The fourth sub-separation processing is processing of replacing, with the pixel value of the arc-shaped connected pixel, a pixel value of a pixel that is surrounded by the arc-shaped connected pixel and is not the arc-shaped connected pixel.

In the separation degree improvement image generation processing, next, fifth sub-separation processing is executed. The fifth sub-separation processing is processing in which the region of the arc-shaped connected pixel, which is generated smaller than an outer shape of the actual tooth root to reliably separate the tooth root and the alveolar bone in the second sub-separation processing, is enlarged by a morphology transformation to be larger than the outer shape of the actual tooth root.

In the separation degree improvement image generation processing, next, sixth sub-separation processing is executed. The sixth sub-separation processing is processing of setting all the pixel values of the slice images not selected in the first sub-separation processing to 0.

Accordingly, the separation degree improvement image generation processing is processing of performing, with reference to the position of the point, the arc-shaped connected point determination processing with a setting of setting the region smaller than the actual tooth root and then enlarging the region. As a result, the second registration can be executed, and the mask can be automatically generated only based on the first designation information and the jaw designation information. Therefore, an image in which the degree of separation between the tooth and the alveolar bone shown in the separation target image is improved is generated by executing the separation degree improvement image generation processing.

Note that the case where the user inputs the first designation information, the second designation information, the point position information, and the jaw designation information is described above as an example. However, the first designation information, the second designation information, the point position information, and the jaw designation information may be stored in the storage unit 14 in advance. For example, when the analysis target image and the comparison target image are selected by the user and the position indicated by the first designation information is substantially the same in any image, the user does not need to input the first designation information.

When images are obtained in which the position indicated by the first designation information is substantially the same in any image due to limitations of an image capturing environment even if the user does not perform the selection, the user also does not need to input the first designation information. The same applies to the second designation information, the point position information, and the jaw designation information.

Not that in the generation of the mask data, range position information may be used instead of the point position information. The range position information may be information indicating a tooth cervical part. Since a lower part of the tooth cervical part is the tooth root, the mask data is generated even if the range position information is used instead of the point position information.

Note that the three-dimensional image processing device 1 does not necessarily have to be implemented by a single housing. The three-dimensional image processing device 1 may be implemented by a plurality of information processing devices communicably connected via a network. In this case, the functional units included in the three-dimensional image processing device 1 may be distributed and installed in a plurality of information processing devices.

The output unit 15 is an example of a predetermined display destination. Note that the first transformed image is an example of the one image after the execution of the first registration processing.

All or some of the functions of the three-dimensional image processing device 1 may be implemented using hardware such as an application specific integrated circuit (ASIC), a programmable logic device (PLD), or a field programmable gate array (FPGA). The program may be recorded on a computer-readable recording medium. The computer-readable recording medium refers to a storage device, for example, a portable medium such as a flexible disk, a magneto-optical disk, a ROM, and a CD-ROM, and a hard disk built in the computer system. The program may be transmitted via an electric communication line.

As described above, the embodiment of the invention has been described in detail with reference to the drawings, however, specific configurations are not limited to the embodiment, and designs and the like within a range not departing from the gist of the present invention are also included.

### Reference Signs List

1 three-dimensional image processing device
11 control unit
12 input unit
13 communication unit
14 storage unit
15 output unit
111 image processing unit
112 input control unit
113 communication control unit
114 storage control unit
115 output control unit
91 processor
92 memory

1. A three-dimensional image processing device for generating image data of a three-dimensional image used for an analysis of a diagnosis target, the three-dimensional image processing device comprising:
a processor;
a storage medium having computer program instructions stored thereon, wherein the computer program instructions, when executed by the processor, perform processing of
executing alignment between a three-dimensional image showing an analysis target captured at a first timing and a three-dimensional image showing the analysis target captured at a second timing different from the first timing, wherein
each of the three-dimensional images includes an image of an object in a preset space including the analysis target,
the diagnosis target is present in the space, and
the analysis target is a support unit, and
the alignment includes:
first registration processing of performing a rigid transformation on one three-dimensional image of the two three-dimensional images to reduce a difference between the one three-dimensional image and the other three-dimensional image of the two three-dimensional images; and
second registration processing of performing a rigid transformation on the one three-dimensional image to reduce a difference between an image of the analysis target shown in the other three-dimensional image and the image of the analysis target shown in the one three-dimensional image after execution of the first registration processing.

2. The three-dimensional image processing device according to claim 1, wherein
in the second registration processing, mask data indicating a pixel of a region that is a region on the other three-dimensional image and includes the image of the analysis target among pixels of the other three-dimensional image is used.

3. The three-dimensional image processing device according to claim 2, wherein
the mask data is image data of a binary image in which an image of an object in a predetermined space including an analysis target and other images have different pixel values.

4. The three-dimensional image processing device according to claim 3, wherein
in the binary image, a pixel value of an arc-shaped connected pixel is one pixel value of two preset pixel values, and a pixel value of a pixel that is not the arc-shaped connected pixel is the other pixel value of the two preset pixel values, wherein a curve is in a three-dimensional image and has, as one end, one predetermined point between an image of the support unit that is the analysis target and an image of a functional unit of a tooth that has the analysis target, the arc-shaped connected pixel is a pixel located at an end of the curve, out of other ends of the curve, which satisfies a condition that a difference between pixel values of all points on the curve and a pixel value of the one predetermined point is within a preset predetermined range, and the three-dimensional image includes the image of the support unit, but does not include the image of the functional unit, and
the functional unit is a tooth crown of a natural tooth, an upper structure of a dental implant, or a head or a plate of an orthopedic implant.

5. The three-dimensional image processing device according to claim 1, wherein the computer program instructions further perform processing of:
controlling an operation of a predetermined display destination,
generating a three-dimensional emphasized display image, which is a three-dimensional image in which a difference between the other three-dimensional image and the one three-dimensional image is indicated by emphasis display based on the other three-dimensional image and the transformed one three-dimensional image transformed by the alignment, and
the display destination to display the three-dimensional emphasized display image.

6. The three-dimensional image processing device according to claim 1, wherein the computer program instructions further perform processing of: ng an operation of a predetermined
an operation of a predetermined display destination,
quantitative information indicating a difference between the other three-dimensional image and the one three-dimensional image by a numerical value based on the other three-dimensional image and the transformed one three-dimensional image transformed by the alignment, and
the display destination to display the quantitative information.

7. A three-dimensional image processing method for generating image data of a three-dimensional image used for an analysis of a diagnosis target, the three-dimensional image processing method comprising:
executing alignment between a three-dimensional image showing an analysis target captured at a first timing and a three-dimensional image showing the analysis target captured at a second timing different from the first timing, wherein
each of the three-dimensional images includes an image of an object in a preset space including the analysis target,
the diagnosis target is present in the space,
the analysis target is a support unit, and
the alignment includes:
first registration processing of performing a rigid transformation on one three-dimensional image of the two three-dimensional images to reduce a difference between the one three-dimensional image and the other three-dimensional image of the two three-dimensional images; and
second registration processing of performing a rigid transformation on the one three-dimensional image to reduce a difference between an image of the support unit shown in the other three-dimensional image and the image of the support unit shown in the one three-dimensional image after execution of the first registration processing.

8. A non-transitory computer readable medium which stores a program for causing a computer to function as the three-dimensional image processing device according to claim 1.
